(19) United States
(12) Patent Application Publication
(10) Pub. No.: US 2014/0255956 A1
(43) Pub. Date: Sep. 11, 2014

(54) DIAGNOSIS OF MYOCARDIAL AUTOIMMUNITY IN HEART DISEASE

(71) Applicants: Myra A. Lipes, Brookline, MA (US); Lizbeth Cornivelli, Medford, MA (US); Venkata Siva Rama Krishnam Raju Gottumukkala, Rockville, MD (US)

(72) Inventors: Myra A. Lipes, Brookline, MA (US); Lizbeth Cornivelli, Medford, MA (US); Venkata Siva Rama Krishnam Raju Gottumukkala, Rockville, MD (US)

(21) Appl. No.: 14/288,269

(22) Filed: May 27, 2014

Related U.S. Application Data
(63) Continuation of application No. 13/703,335, filed on Jan. 22, 2013, now abandoned.
(60) Provisional application No. 61/353,530, filed on Jun. 10, 2010.
(60) Provisional application No. 61/358,503, filed on Jun. 25, 2010.

(51) Int. Cl. G01N 33/543

(52) U.S. Cl. CPC G01N 33/54306
USPC 435/7.92; 436/501; 424/172.1; 424/133.1

(57) ABSTRACT

Provided herein are, inter alia, methods of diagnosing myocardial autoimmunity in subjects by detecting the presence of autoantibodies to cardiac antigens in the subjects.

### CLAIM OF PRIORITY

This application claims the benefit of U.S. Provisional Patent Applications Ser. Nos. 61/353,530, filed on Jun. 10, 2010, and 61/358,503, filed on Jun. 25, 2010, the entire contents of which are hereby incorporated by reference.

### FEDERALLY SPONSORED RESEARCH OR DEVELOPMENT

This invention was made with Government support under Grant No. 5RO1 DK072090-05 awarded by the National Institutes of Health. The Government has certain rights in the invention.

### TECHNICAL FIELD

This invention relates to methods of diagnosing myocardial autoimmunity (e.g., a cardiac autoimmune response), e.g., in subjects following myocardial infarction, subjects with unexplained heart failure, or genetically susceptible subjects without clinical heart disease.

### BACKGROUND

Ischemic heart disease (e.g., coronary artery disease or coronary heart disease) refers to heart problems caused by narrowed heart arteries. This can ultimately lead to heart attack (myocardial infarction (MI)). Ischemic heart disease is a leading cause of death in the United States. Patients with type 1 diabetes (T1 D) suffer excessive mortality following an MI. However, the underlying mechanisms arc poorly understood and are not fully explained by conventional cardiovascular risk factors.

### SUMMARY

The present invention is based, at least in part, on the discovery of the presence of persistent autoantibodies to one or more cardiac antigens in subjects who developed an autoimmune response after they have had ischemic heart disease. These studies suggest a novel role of autoimmunity in the pathogenesis of cardiovascular complications in subjects with an autoimmune disease such as T1D. The assays described herein can be used for diagnosis and selection of therapy for autoimmune heart disease in subjects with autoimmune diseases such as T1D and other autoimmune conditions. The serological detection of autoimmune heart markers could guide the use of MRI techniques to confirm myocardial inflammation and the implementation of immune-based therapies (e.g., rituximab) to target these pathways.

Provided herein are methods for diagnosing an autoimmune response following ischemic heart disease in a subject, the method comprising: providing a sample comprising serum of a subject who has suffered ischemic heart disease; and detecting the presence or absence in the sample of autoantibodies to α-actinin-2, wherein the presence of autoantibodies to α-actinin-2 indicates that the subject has an autoimmune response following ischemic heart disease.

In some embodiments, the subject has an autoimmune disorder. In some embodiments, the subject has type 1 diabetes.

The methods described herein can comprise detecting the presence or absence of autoantibodies to cardiac myosin. The method can also further comprise detecting the presence or absence of autoantibodies to troponin. In some embodiments, the presence or absence of autoantibodies to α-actinin-2, cardiac myosin, and troponin is detected. In some embodiments, the presence or absence of autoantibodies that recognize an epitope within subfragment 1 (S1) or the head region of cardiac myosin is detected.

In another aspect, described herein are methods for diagnosing an autoimmune heart disease in a subject, the method comprising: providing a sample comprising serum of a subject who has a heart disease; and detecting the presence or absence in the sample of autoantibodies that bind to an epitope within subfragment S1 of cardiac myosin, wherein the presence of the autoantibodies indicates that the subject has an autoimmune heart disease. In some embodiments, the subject has cardiomyopathy or myocarditis.

In another aspect, the invention provides methods for diagnosing the presence of myocardial autoimmunity in a subject. The methods include providing a sample comprising serum of a subject; and detecting the presence in the sample of one or both of: autoantibodies that bind to alpha-actinin-2 (aActn2), and autoantibodies that bind to cardiac myosin, e.g., to one or more fragments of myosin, e.g., to the S1 fragment of alpha-Myosin Heavy Chain (alpha-MHC-S1), and diagnosing the subject with myocardial autoimmunity based on the presence of autoantibodies to aActn2 and/or alpha-MHC-S1.

In a further aspect, the invention provides methods for selecting a treatment for a subject. The methods include providing a sample comprising serum of a subject; and detecting the presence or absence in the sample of one or both of: autoantibodies that bind to alpha-Actinin-2 (aActn2), and autoantibodies that bind to the S1 fragment of alpha-Myosin Heavy Chain 6 (alpha-MHC6-S1), wherein the presence of autoantibodies to aActn2 and/or alpha-MHC-S1 indicates that the subject has autoimmune myocarditis; and selecting an immune-modulatory treatment for the subject who has autoimmune myocarditis. In some embodiments, the treatment comprises administration of immunomodulatory therapies.

In yet another aspect, the invention features methods for providing a prognosis or predicting risk of mortality in a subject who has ischemic heart disease. The methods include detecting the presence of autoantibodies that bind to alpha-Myosin Heavy Chain 6 (MyHC6); and detecting the presence of autoantibodies that bind to alpha-Myosin Heavy Chain 7 (MyHC7); wherein the presence of autoantibodies that bind to MyHC6 and autoantibodies that bind to MyHC7 indicates that the subject has a poorer prognosis or an increased risk of mortality as compared to a subject who does not have said autoantibodies.;

In some embodiments, the methods also include detecting the presence of autoantibodics to cardiac troponin 1.

In some embodiments, the subject has an autoimmune disorder, e.g., type 1 diabetes or hypothyroidism. In some embodiments, the subject has had ischemic heart disease, e.g., myocardial infarction (MI) or coronary artery disease (CAD).

In some embodiments, the subject experienced the ischemic heart disease at least one month prior to the provision of the sample, e.g., at least one year prior to the provision of the sample.

In some embodiments, the methods further include confirming the presence of myocarditis, e.g., by cardiac imaging.

In some embodiments, the methods further include administering an immunomodulatory therapy, e.g., administration of rituximab or anti-CD3 antibody.

In another aspect, the invention provides kits for use in the methods described herein, comprising one or more of the following panels of human autoantigens:

(i) Actn2 and TnI;

(ii) full-length MyHC6 and full-length MyHC7;

(iii) MyhC6-S1, MyhC6-S2, and MyhC6-LMM;

(iv) MyhC6-S1, MyhC6-S2, MyhC6-LMM full-length MyHC6, and full-length MyHC7; and/or

(v) Actn2, TnI, and one or more of MyhC6-S1, MyhC6-S2, MyhC6-LMM full-length MyHC6, and/or full-length MyHC7,

or any combination thereof.

In some embodiments, the autoantigens are labeled, e.g., radiolabeled.

In some embodiments, the kits further a reagent for detecting or purifying autoantigen/antibody complexes.

In some embodiments, the reagent comprises one or both of protein A and protein G, e.g., bound to a bead.

The term “subject” is used throughout the specification to describe an animal, human or non-human, to whom treatment according to the methods of the present invention is provided. Veterinary and non-veterinary applications are contemplated. The term includes, but is not limited to, mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a set of serial sections showing lymphocytic infiltrates adjacent to the infarct zone only in NOD mice (left panel, boxes). IHC staining showing that the cardiac infiltrates (middle panel) consist of B220 cells, CD4⁺ and CD8⁺ T cells, similar in composition to “insulitis” lesions (right panel).

FIG. 1B is a set of images showing extension of the infiltrates and poor healing in a NOD mouse heart 8 wk post-MI (upper panel).

FIG. 2A is an immunoblot analysis showing that the autoantibodies from post-MI NOD mice (but not non-diabetic B6 mice) are reactive to myosin heavy chain (MyHC; arrow) and Actn2.

FIG. 2B is a set of Western blots showing that serum from post-MI NOD mice recognizes in vitro translated and purified recombinant Actn2 identically to the native Actn2 contained in heart lysates (He).

FIG. 3 is a set of three dot plots showing the prevalence of cardiac autoantibodies in sera from post-MI T1D and T2D patients and healthy control (HC) subjects. Cardiac autoantibodies were measured with radioimmunoprecipitation assays using recombinant human Actn2, troponin I and overlapping fragments of α-myosin (the S1 fragment is shown). The dashed lines indicate the mean+3SD of the normal control group; values above this are considered positive. H-67 is a T2D patient who tested positive for both α-myosin and troponin 1 autoantibodies.

FIGS. 4A-F are cardiac magnetic resonance images (MRI) showing evidence of myocardial inflammation (“myocarditis”) in a 61-yr-old post-MI type 1 diabetic patient with unexplained rapidly progressive heart failure, who tested positive for α-myosin (S1) autoAbs. Cine imaging (4A) showing dilated hypocontractile left ventricle with bilateral pleural effusion as a result of heart failure. There is evidence of chronic myocardial inflammation by T2-weighted fast spin-echo edema imaging (4B, arrows). In addition, myocardial inflammation was observed comparing T2* images before (4C and 4E, at echo times of 1.4 and 21 msec, respectively) and after (4D and 4F, at echo times of 1.4 and 21 msec, respectively) injection of the iron-oxide agent, Ferumoxytol. At 24 hr after injection, there was evidence of accumulation of iron-oxide in the myocardium, indicated by the decay constants of T2*

FIGS. 4G and H are decay curves from the same patient as in 4A-F, taken 24 msec from before vs. 9.4msec after iron-oxide injection.

### DETAILED DESCRIPTION

Described herein are methods for diagnosing autoimmunity following ischemic heart disease in subjects by detecting the presence or absence of autoantibodies to one or more cardiac antigens.

Myocardial infarction is known to induce a profound inflammatory response with the influx of monocytes/macrophages and production of proinflammatory cytokines that are crucial for cardiac repair and resolve with tissue healing. It was not known whether these same inflammatory pathways might exert “adjuvant effects” and lead to aberrant immune responses in subjects.

Data described herein show that experimental induction of myocardial infarction (MI) by coronary artery ligation in 7-8 week old normoglycemic non-obese diabetic (NOD) mice, but not in age-matched control C57BL/6 mice, triggered rapid (within 1 week) development of an irreversible post-infarct autoimmunity syndrome characterized by: 1) sustained production of high-titer IgG autoantibodies targeted against cardiac myosin and the cardiac Z-disk protein, α-actinin-2; 2) destructive lymphocytic infiltrates in the myocardium, similar in composition to pancreatic insulitis lesions; and 3) poor infarct healing. Data also demonstrate that myocardial ischemic injury induces autoimmunity in human patients with T1D.

A novel ischemia-specific autoantigen, α-Actinin-2 (Actn2), in post-infarction autoimmunity (PIA) was identified. Data show that Actn2 autoantibodies were present in a significant portion of the post-infarcted T1D patients tested.

Data also demonstrate that testing for autoantibodies to multiple cardiac antigens (e.g., Actn2, cardiac myosin (alpha-MyHC, in particular, the S1 fragment of alpha-MyHC) and troponin I) has high positive predictive value for autoimmunity following myocardial infarction.

These findings suggest that cardiac autoimmunity contributes to worsened post-MI outcomes in patients. Thus, it may be useful to diagnose autoimmunity following ischemic heart disease in a subject, so that appropriate immunomodulatory treatments can be administered. Accordingly, provided herein are methods for diagnosing autoimmunity following ischemic heart disease in a subject. The methods include detecting the presence of autoantibodies to one or more cardiac antigens (e.g., Actn2, myosin, and troponin I) in a subject who has suffered ischemic heart disease. The presence of antibodies to one or more cardiac antigens indicate that the subject is suffering from autoimmunity following ischemic heart disease.

α-Actinin-2

α-Actinin-2 (Actn2) is a 105 kDa structural protein. Actn2 is the main component of sarcomeric Z-bands where it functions to anchor actin-containing thin filaments together. Although the primary function of Actn2 is in actin binding, over 20 other different binding partners have been discovered so far, including inducible NO synthetases, P1-3 kinascs, B1 integrins and cardiac ion channels.

Actn2 polypeptides and antigenic fragments thereof and nucleic acids encoding Actn2 polypeptides and antigenic fragments thereof are useful in the methods described herein. Exemplary Actn2 amino acid sequences can be found at Genbank Accession Nos. NP_{—}001094.1 (human) and NP_{—}150371.4 (mouse). Exemplary Actn2 nucleic acid sequences can be found at Genbank Accession Nos NM_{—}001103.2 (human), M86406.1 (human), AY036877.1 (mouse) and NM_{—}033268.4 (mouse).

A nucleic acid encoding a mammalian, e.g., human, Actn2 amino acid sequence can be amplified from human cDNA by conventional PCR techniques, using primers upstream and downstream of the coding sequence. Vectors containing full-length human Actn2 cDNAs are also commercially available from OriGene.

One method for producing Actn2 polypeptides for use in the invention is recombinant production, which involves genetic transformation of a host cell with a recombinant nucleic acid vector encoding an Actn2 polypeptide, expression of the recombinant nucleic acid in the transformed host cell, and collection and purification of the Actn2 polypeptide. Guidance concerning recombinant DNA technology can be found in numerous well-known references, including Sambrook et al., 2001, “Molecular Cloning—A Laboratory Manual,” 3d Ed. Cold Spring Harbor Press; and Ausubel et al. (eds.), 2002, “Short Protocols in Molecular Biology,” John Wiley & Sons, Inc.

Purification of recombinant Actn2 polypeptides can be performed by conventional methods and is within ordinary skill in the art. The purification can include two or more steps, and one step can be affinity chromatography employing anti-Actn2 antibodies covalently linked to a solid phase chromatography support (beads) such as crosslinked agarose or polyacrylamide. Other useful purification steps include gel filtration chromatography and ion exchange chromatography.

Cardiac Myosin

Myosin is a large family of motor proteins. It is a hexameric protein containing two heavy chain subunits, and four light chain subunits. Cardiac myosin is a major autoantigen in numerous autoimmune heart conditions. Cardiac myosin refers to an isoform of myosin expressed in cardiac muscles. For example, myosin heavy chain α (MyHCα) and is a cardiac-specific isoform. When myosin is exposed to the proteolytic enzyme trypsin, fragmentation occurs to yield heavy meromyosin (HMM) and light meromyosin (LMM). HMM containing the head and a short tail can be further split by proteolytic enzymes, such as papain, into subfragment 1 (S1) and subfragment 2 (S2). In some embodiments, the methods include detecting antibodies that bind to S1.

MyHCα polypeptides and antigenic fragments thereof and nucleic acids encoding MyHCα polypeptides and antigenic fragments thereof are useful in the methods described herein. Exemplary MyHCα amino acid sequences can be found at, e.g., Genbank Accession Nos. NP_{—}002462.2 (human) and NP_{—}034986.1 (mouse). The various domains within MyHCα are known in the art, e.g., residues 88-768 of the amino acid sequence of NP_{—}002462.2 contain the head region. Exemplary MyHCα nucleic acid sequences can be found at Genbank Accession Nos NM_{—}002471.3 (human) and NM 010856.4 (mouse).

Cardiac myosin and immunogenic fragments thereof and nucleic acids encoding cardiac myosin and fragments thereof can be generated using the methods known in the art or described above. Cardiac myosin can also be purified from cardiac tissues (e.g., from human or mouse) using methods known in the art. See, e.g., Caforio et al., Circulation, 1992, 85:1734-1742. Subfragment 1(S1) of myosin, which includes the globular head region of the myosin molecule, can be prepared from chymotryptic or papain digests of myosin.

Troponin

Troponin has three subunits, troponin C (TnC), troponin I (TnI), and Troponin T (TnT). Troponin is used as a diagnostic markers for heart disorders. For example, TnT is an intracellular, compartmentalized protein—a myofilament component within the myofiber, and its release to the circulation occurs after loss of membrane integrity, irreversible cell damage and reperfusion (Van Eyk et al., Circ Res. 1998; 82:261-71). Release of TnI from myocardium is usually detected in circulation 4-6 hours after infarction, and the duration of the TnT cycle is about 3-10 days (Beckett et al., Cardiovascular disorders. In Lecture Notes: Clinical Biochemistry, 7^{th} ed. Oxford, UK:Blackwell Publishing, Ltd., 2005. Ch 11, pp 160-76).

TnI polypeptides and nucleic acids encoding TnI polypeptides are useful in the methods described herein. Exemplary TnI amino acid sequences can be found at, e.g., Genbank Accession Nos. NP_{—}000354.4 (human) and NP_{—}033432.1 (mouse). Exemplary TnI nucleic acid sequences can be found at Genbank Accession Nos NM_{—}000363.4 (human) and NM_{—}009406.3 (mouse).

TnI polypeptides and nucleic acids encoding TnI polypeptides can be generated using the methods known in the art or described above.

Diagnostic and Prognostic Methods

Provided herein are methods for diagnosing autoimmunity following ischemic heart disease in a subject. The methods include detecting the presence or absence of autoantibodics to one or more cardiac antigens (e.g., Actn2, cardiac myosin, and troponin) in a subject who has suffered ischemic heart disease. The presence of autoantibodies to one or more cardiac antigens described herein indicate that the subject is suffering from autoimmunity following ischemic heart disease.

In some embodiments, the presence or absence of autoantibodies to troponin I is detected. In some embodiments, the presence or absence of autoantibodies to an epitope within subfragment 1 of cardiac myosin is detected.

In some embodiments, a subject is tested for the presence or absence of autoantibodies to a panel of cardiac antigens, e.g., Actn2, cardiac myosin, and troponin I. Data described herein demonstrate that a high percentage of post-MI T1D patients tested were positive for autoantibodies to at least one cardiac antigens. Thus, testing for autoantibodies to a panel of cardiac antigens is expected to more accurately identify autoimmunity in patients who have had ischemic heart disease.

The data presented herein also show that the presence of autoantibodies to isoforms or fragments of myosin, e.g., the S1 domain of cardiac myosin, is characteristic of myocardial autoimmunity. Antibodies to the alpha and beta isoform (MYH6 and MYH7, respectively) are also characteristic of autoimmune myocarditis, and the presence of autoantibodies to multiple isoforms, i.e., to both the alpha and beta isoform (MYH6 and MYH7), is indicative of a poorer prognosis, e.g., an increased risk or rate of mortality. Thus, methods for diagnosing and prognosing autoimmune heart diseases are also provided.

Once it has been determined that a subject is suffering from myocardial autoimmunity, the information can be used in a variety of ways. For example, a decision to administer a specific immunomodulatory treatment or to treat more aggressively can be made.

The methods described herein are useful in a wide variety of clinical contexts. For example, the methods can be used for diagnosing subjects in hospitals and outpatient clinics, as well as the Emergency Department. The methods can be carried out on-site or in an off-site laboratory.

Detecting Autoantibodies

Methods known in the art or described herein can be used to detect the presence or absence of autoantibodies to the cardiac antigens described herein. Generally, scrum samples from subjects are contacted with the cardiac antigens described herein, for a sufficient amount of time and under conditions that allow binding of the cardiac antigens to any autoantibodies in the serum samples. Binding between the cardiac antigens and the autoantibodies are then detected and, in some embodiments, quantified.

For example, enzyme-linked immunosorbent assay (ELISA) can be used to detect the presence of autoantibodies in the serum of subjects. ELISA can detect autoantibodies that bind to antigens immobilized on solid support (e.g., a multi-well plate) by using enzyme-linked secondary antibodies, such as goat anti-human Ig Abs, and enzyme substrates that change color in the presence of enzyme-labeled antibodies.

In preferred embodiments, fluid-phase assays such as radioimmunoassays (RIA) can also be used to detect the presence of autoantibodies. For example, the gene for the antigen (or a fragment thereof) can be cloned into an expression vector, and in vitro translation can be carried out with [³⁵S]methionine to produce radiolabeled antigens. Antibody-bound radiolabeled antigens can be separated from free radiolabeled antigens with, e.g., protein A-Sepharose or protein G-Sepharose beads, which bind to the antibodies. The presence of antibodies in a sample can be detected using methods known in the art and described herein.

Subject Population

The diagnostic methods describe herein can be used to diagnose autoimmunity following ischemic heart disease in any subject who has had ischemic heart disease. The methods are suitable for diagnosing subjects who suffered an heart ischemic event shortly (e.g., one week to a few months) or some time (e.g., up to 12 years) prior to the application of the methods described herein. Thus, the methods can be applied any time after a subject had suffered ischemic heart disease. In some cases, it might be beneficial to test the presence of autoantibodies to one or more cardiac antigens in a subject no earlier than 1 month after a cardiac event to avoid false positives (e.g., transient autoimmunity).

In some embodiments, the subject has (i.e., has been diagnosed with) an autoimmune disorder. In some embodiments, the subject has (i.e., has been diagnosed with) type 1 diabetes or hypothyroidism. In some embodiments, the methods include selecting the subject on the basis that they have or have been diagnosed with an autoimmune disorder, e.g., type 1 diabetes or hypothyroidism.

In one aspect, the methods can be used to diagnose autoimmunity in subjects who have heart disorders such as myocarditis and idiopathic cardiomyopathy (unexplained heart failure), or genetically susceptible subjects without clinical heart disease.

As used herein, an autoimmune disorder is a condition that occurs when the immune system mistakenly attacks and destroys healthy body tissue. Examples of autoimmune (or autoimmune-related) disorders include, but are not limited to type 1 diabete mellituss, thyroiditis (e.g., Hashimoto's thyroiditis or Ord's thyroiditis), pernicious anemia, Addison's disease I, rheumatoid arthritis, systemic lupus erythematosus (SLE), dermatomyositis, Sjogren syndrome, lupus erythematosus, multiple sclerosis, myasthenia gravis, reactive arthritis, Grave's disease, celiac disease, Crohn's disease, acute disseminated encephalomyelitis, ankylosing spondylitis, antiphospholipid antibody syndrome, aplastic anemia, autoimmune hepatitis, autoimmune oophoritis, celiac disease, Goodpasture's syndrome, Guillain-Barré syndrome, idiopathic thrombocytopenic purpura, Kawasaki's disease, opsoclonus myoclonus syndrome, optic neuritis, pemphigus, polyarthritis, primary biliary cirrhosis, psoriasis, Reiter's syndrome, small vessel vasculitis, Takayasu's arteritis, temporal arteritis, ulcerative colitis, warm autoimmune hemolytic anemia, or Wegener's granulomatosis idiopathic membranous nephropathy.

A diagnosis of an autoimmune disorder, e.g., of Type 1 diabetes, can be made by a clinician using methods known in the art.

Methods of Treatment

The methods described herein include methods for the treatment of autoimmune myocarditis. Generally, the methods include administering a therapeutically effective amount of an immune suppressive treatment to a subject who has been determined to be in need of such treatment by a method described herein.

As used in this context, to “treat” means to ameliorate at least one symptom or clinical sign of autoimmune myocarditis. Often, autoimmune myocarditis results in impaired cardiac pumping function or arrhythmias; a treatment can result in a reduction in cardiac inflammation and a return or approach to normal cardiac function or rhythm.

In some embodiments, the methods also include confirming the presence of myocarditis, e.g., by cardiac imaging using known methods such as MRI or biopsy to detect evidence of inflammation. The presence can be confirmed before administration of a treatment.

Immune suppressive treatments are known in the art and include those treatments that remove activated T cells. Exemplary treatments include administration of rituximab; anti-CD3 antibodies; IFN-alpha; IV immunoglobulin; immunoabsorption therapy; azathioprine; thymomodulin; tacrolimus; sirolimus; mycophenolate; fingolimod; and myriocin. Non-diabetic subjects may be treated with immune suppressive glucocorticoinds such as prednisone and cyclosporine. See also Rose and Baughman, “Myocarditis and dilated cardiomyopathy.” In: Rose and Mackay, eds. The Autoimmune Diseases. (Boston, Mass., USA: Academic Press; 2006:875-888).

Kits

Also provided herein are kits for use in the methods described herein, including one or more antigens, and optionally one or more antibodies. In some embodiments, the kit includes antigens, e.g., recombinantly produced antigens, for use in a method described herein. For example, the kits can include Actn2, TnI, and MyHC antigens. The kits may contain one or more isoforms of MyHC, e.g., MyHC6 and MyHC7. In some embodiments the kits contain one or more fragments of MyHC, e.g., S1, S2, and LMM, e.g., fragments of MyHC6. The antigens can be produced using methods known in the art, e.g., by in vitro translation or production from cells that express exogenous antigen, e.g., a cultured cell line or transgenic animal expressing the antigen. In some embodiments, the antigens are produced from a human cell line. In some embodiments, the fragments of MyHC are produced by proteolytic digestion of full-length MyHC. Preferably, the antigens are human, e.g., human sequences.

The antigens can be provided for use, e.g., in lyophilized form, in solution, or bound to a substrate, e.g., a solid surface such as an array, or beads, e.g., microspheres.

In some embodiments, the antigens are labeled, e.g., with a detectable moiety. The label can be or include, e.g., various enzymes, prosthetic groups, fluorescent materials, luminescent materials, bioluminescent materials, and radioactive materials.

Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, quantum dots, or phycoerythrin; an example of a luminescent material includes luminol; examples of bioluminescent materials include luciferase, luciferin, and aequorin, and examples of suitable radioactive materials include ¹²⁵S, ¹³¹I, ³⁵S or ³H. The detectable moiety must not interfere with binding of autoantibodies to the antigens.

In some embodiments, the kits can also include reagents for the purification or detection of autoantibodies that bind to the antigens, e.g., protein A or protein G, or anti-human antibodies. In some embodiments, the reagents are bound to a substrate, e.g., a solid substrate or beads.

In some embodiments, the kits can include antibodies to Actn2, TnI, and MyHC. The kits may contain antibodies that bind specifically to one or more isoforms of MyHC, e.g., MyHC6 and MyHC7. In some embodiments the kits contain antibodies that bind specifically to one or more fragments of MyHC, e.g., S1, S2, and LMM, e.g., fragments of MyH6C. Such antibodies can be used, e.g., as standards or controls, and can be human antibodies (e.g., recombinant or chimeric human antibodies).

### EXAMPLES

### Example 1

### Establishment of a Preclinical Mouse Model of Myocardial Infarction-Induced Autoimmunity

Described in this example is the establishment of an experimental model that can permit detailed mechanistic studies on how autoimmune reactions may contribute to cardiovascular disease complications in individuals with type 1 diabetes. Acute MI was experimentally induced by occluding the left anterior descending coronary artery in normoglycemic 7-8 wk-old NOD and control B6 mice, and the mice were followed for up to 12 weeks. To optimize survival, infarcts were induced that involved on average 20-30% of the left ventricle and were not extensive enough to result in cardiac failure.

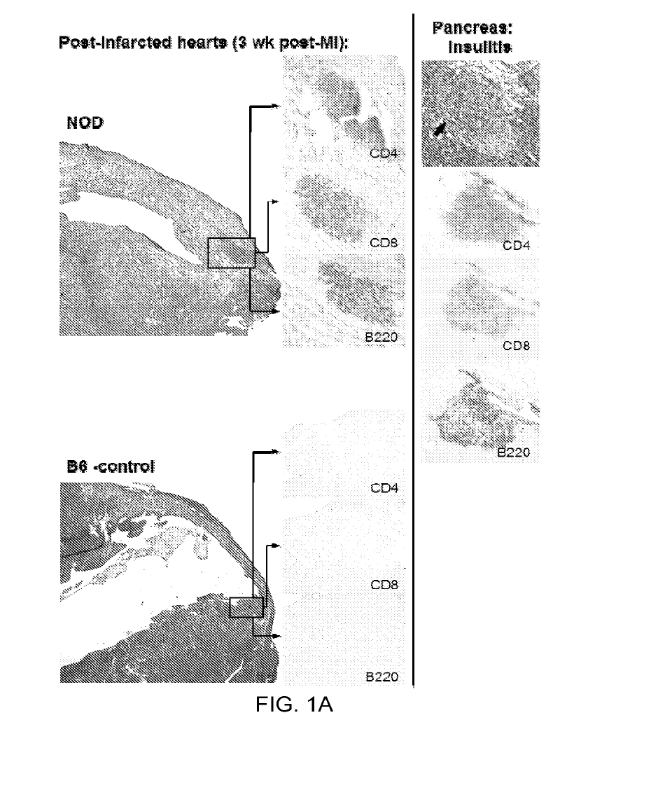

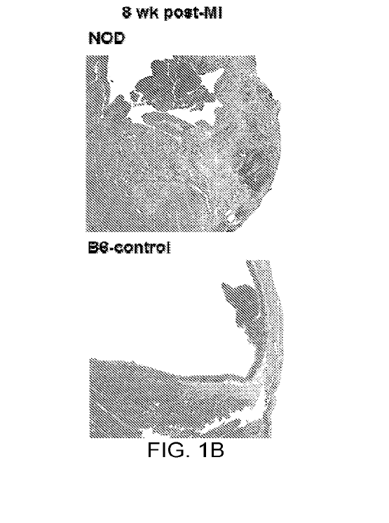

The mice were evaluated for the presence of autoantibodies to cardiac antigens as previously described (Taylor et al., J Immunol. 2004 Feb. 15; 172(4):2651-8; Lv et al, J. Clin Invest. 2011;121(4):1561-1573). As expected, none of the unmanipulated control NOD mice tested positive for cardiac autoantibodies at baseline or during the course of the study. However, starting as early as 1 week after infarction, NOD mice—but not B6 mice—developed high-titer circulating IgG autoantibodics which, by indirect immunofluorescence and confocal microscopy on healthy heart tissue, localized to the striations within myocytes, producing a distinctive myofibrillar pattern, similar to that observed in serum from HLA-DQ8 transgenic mice with spontaneous autoimmune myocarditis (Taylor et al., 2004). Remarkably, direct immunofluorescence analysis revealed diffuse deposition of IgG antibodies over the entire heart, including regions remote from the infarct zone in the post-infarcted NOD hearts, whereas the post-infarcted B6 hearts were devoid of IgG deposition. Moreover, post-MI NOD mice developed lymphocytic infiltrates in the heart, with a cellular composition (CD4+ and CD8+ T cells, and B220+ B cells; see FIG. 1A) that closely mirrored insulitis lesions in the pancreas (FIG. 1B). In addition, longer follow-up of post-MI NOD showed that PIA was associated with impaired infarct healing.

In this model, the severity of post-infarction autoimmunity (PIA) correlated with the size of the initial cardiac injury. The severity of PTA also correlated with the specific location of the occluding suture, with the “high” suture location (near the atrioventricular junction, at the edge of the atrial appendage) inducing the most robust PIA phenotype, but still enabling a ˜50% survival. In contrast, NOD mice did not develop antibodies or myositis in response to acute necrotic skeletal muscle injuries known to stimulate muscle regeneration: cold injury (dry ice), mechanical injury (crush injury) and chemically-induced injury (cardiotoxin). These results demonstrate that the induction of PIA was specific to ischemic heart injury and was not part of a generalized response to tissue injury.

### Example 2

### Identification of a Novel Ischemia-Specific Autoantigen, α-Actinin-2, in PIA

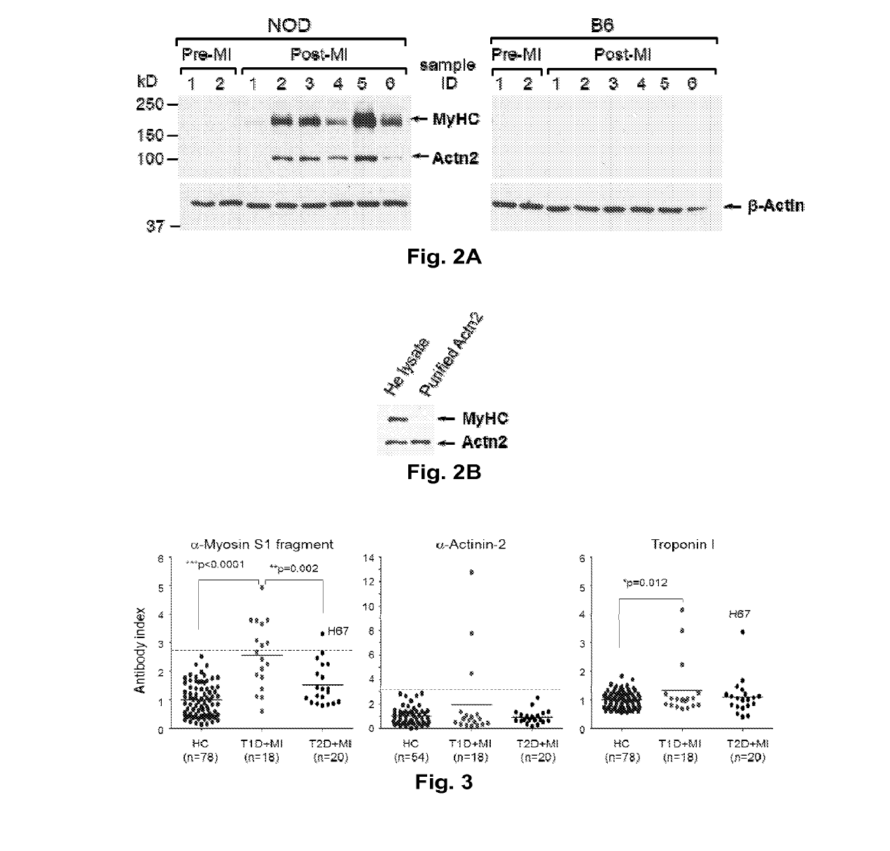

Although acute necrotic injury from MI might have been expected to result in the exposure and loss of tolerance to multiple autoantigens, we found that post-MI NOD mice developed autoantibodies to predominantly two proteins: myosin heavy chain (MyHC) and a second ˜105 kDa protein. This protein was only detectable in SDS (Laemmli) lysates and was not present in the myofibrillar heart extracts (“MFE”) that were previously used for Western blot analysis (FIG. 2A). The ˜105 kDa protein was expressed at high levels in cardiac muscle and skeletal muscle but was absent in lung, liver, kidney and brain Immunoprecipitation of this protein with serum from post-MI NOD mice, followed by excision and enzymatic digestion of the band from a gel, and analysis by tandem mass spectrometry revealed that its peptide sequences were identical to the 105 kDa structural/cytoskeletal protein, α-actinin-2 (hereafter, Actn2), which has a MW of 103,854 kDa. Actn2 is the main component of sarcomeric Z-bands where it functions to anchor actin-containing thin filaments together. Although the primary function of Actn2 is in actin binding, over 20 other different binding partners have been discovered so far, including inducible NO synthetases, PI-3 kinases, B1 integrins and cardiac ion channels.

Interestingly, antibodies against Actn2 were detectable as early as 1 wk post-MI (the earliest timepoint examined) but myocarditis mice did not develop Actn2 autoantibodies until very late in the disease course, once the mice had signs of severe congestive heart failure. These results suggested that the development of Actn2 autoantibodies was ischemic cardiac injury-specific. Importantly, the autoantibodies to cardiac myosin and Actn2 remained elevated for at least 12 wk after MI (the longest the mice were followed).

Interestingly, NOD mice also developed autoantibodies to cardiac myosin and Actn2 after smaller infarctions (‘microinfarctions’), produced by placing a suture around the left coronary artery without permanent ligation. These manipulations resulted in focal areas of myocardial fibrosis by Masson's trichrome staining rather than the widespread necrosis characteristic of permanent ligation. However, the prevalence and titers of cardiac autoantibodies after microinfarction was lower than those observed after full-scale MI, with <50% of microinfarcted NOD mice (6/15) exhibiting positive cardiac autoantibody titers at the end of the study period. These findings suggested that the severity of PIA correlated with the magnitude of the initial cardiac injury. The sham-operated NOD mice that received just open-chest thoracotomy, but no LAD coronary occlusion, did not develop autoantibodies or cardiac infiltrates. Thus, the development of PIA was strictly due to myocardial injury and did not result from the nonspecific effects of open-chest surgical trauma or anesthesia (Entman et al., 2000).

### Example 3

### Cloning, Expression, and Purification of Mouse Actn2

To confirm the specificity of Actn2 autoantibodies, recombinant mouse Actn2 was produced using known techniques in Esherichia coli as a histidine-tagged fusion protein, followed by purification. This was accomplished by PCR-cloning the cDNA of Actn2 from NOD mouse heart mRNA, subcloning the mouse Actn2 cDNA into the pET20b expression vector (Novagen) and performing sequencing to confirm that no PCR errors had been introduced. This construct, containing a histidine-tagged C-terminus fusion protein of mouse Actn2, was used to transform BL21 DE3 E. coli cells (Novagen). Protein expression was induced by the addition of IPTG (FIG. 2B) and cells were collected by centrifugation 4 h following induction. The His₆-tagged Actn2 protein was purified over a Ni²⁺-charged chelating Separose fast flow resin column (Amersham), followed by dialysis and further purification by HPLC with a HiLoad 16/60 Superdex 200 prep grade anion exchange column (GE Healthcare), with the purity of Actn2 verified by SDS-PAGE. Subsequent immunoblot assays confirmed that sera from post-MI NOD mice detected purified recombinantActn2 protein in an identical manner to native Actn2 in heart lysates.

With this improved source of mouse Actn2 antigen, an Actn2 enzyme-linked immunoadsorbent assay (ELISA) was developed that enabled more accurate quantitation of the titers of Actn2 autoantibodies in post-MI NOD mice. These studies confirmed that NOD mice with PIA developed high-titer autoantibodies against both Actn2 and cardiac myosin.

### Example 4

### Discovery of a PIA Syndrome in T1D Humans

The development of a cardiac autoimmunity syndrome in post-infarcted NOD mice raised the possibility that some human subjects with T1D might also develop cardiac autoimmunity following MI. Since cardiac autoantibodies developed very soon after infarction in the NOD mouse model (1 week, the earliest time-point examined) and remained persistently elevated in post-MI NOD mice, this suggested that the timing of sample collection relative to the date of the MT might not be critical.

Although the presence of autoantibodics to cardiac myosin and Actn2 could be clearly distinguished between pre- and post-infarcted NOD mice using Western blot and ELISA techniques, human serum did not perform as well in the solid-phase (ELISA) assay formats that were used, with unacceptable numbers of normal control subjects showing false-positivity. Modeling on the success of “biochemical” islet autoantibody assays that are widely used in T1D screening programs, fluid-phase cardiac autoantibody assays were developed using ³⁵S-methionine-labeled in vitro transcribed and translated recombinant human cardiac proteins followed by precipitation with protein-A/G Sepharose beads (Stewart et al., Circulation.2010; 122: A17040).

Subjects consisted of 18 consecutively recruited post-MI with T1D, mean age 56±12 years (range 19-68 years), 53% (9/17) females with a mean time interval from

MT to autoantibody testing =4.4 years (range 0.5-8 years); and, as controls, 20 consecutively recruited T2D post-MI patients, 75% males (15/20), mean age 60±11 years (range 36-80 years) with mean time interval from MT to antibody testing=9.8 years (range 0.6-24 years).

Human Cardiac Autoantigen Radioimmunoassay (RIA)

The cardiac autoantigens included human Actn2, alpha-MyHC (“cardiac myosin”), and cardiac troponin T, which has also been implicated as an important cardiac autoantigen. Because of the large size of a-MyHC, assays were established with three overlapping fragments encompassing the entire protein (S1, S2, LMM). Overlapping fragments of nucleic acid encoding the subfragment 1 (S1), subfragment 2 (S2), and light meromyosin (LMM) domains of human MYH6 (cardiac myosin heavy chain alpha) were amplified by PCR using the primers shown in Table 1, and cloned. The S1 polypeptide used in this example corresponds approximately to the first 865 amino acids of human MYH6 (e.g., Genbank Accession No. NP_{—}002462.2).

TABLE 1
Primers used to generate MYH6 cDNA fragments
by PCR amplification

| | | SEQ |
|---|---|---|
| Frag- | | ID |
| ment | Sequence | NO: |
| S1 | F: TTGCACTCGAGAATTCCGAGATGACCGATGCCCAGATGG | 1 |
| | R: TACCACGCGTGCGGCCGCTCACAGCGTCTCTTTGATGCG | 2 |
| S2 | F: TTGCACGTCGACACCATGGCCTTCATGGGGGTCAAG-3′ | 3 |
| | R: TACCACTGCGGCCGCTCACGCCTTGCCCTCCTCCTCCAG | 4 |
| LMM | F: TTGCACGTCGACAACATGGAGCAGATCATCAAGGCC- | 5 |
| | R: TACCACGCGTGCGGCCGCAGGTTCCCGAGGCAGTGTCAC | 6 |

Each cDNA (or fragment, in the case of a-MyHC) was cloned into an expression vector (e.g., pCMV-TnT, Promega). All plasmid clones were sequenced completely to verify that no sequence errors had been introduced and also to confirm the orientation of the clone in order to express in vitro either from T7 or SP6 promoter. The autoantigens were in vitro translated with [³⁵S]methionine as follows. Plasmid DNA (2 μg) was incubated in a 40 μl of TnT T7 or SP6 quick coupled transcription/translation (Promega, Madison, USA) with 2 μl of [³⁵S]-methionine (1000 Ci/mmol; 10 mci/ml; GE Healthcare, Piscataway, USA) and made up the total volume of the reaction to 50 μl with nuclease free water. The reaction was incubated for 90 minutes at 30° C. Gel electrophoresis was used to confirm that the translated product resulted in protein band of the appropriate size. Patient samples were screened in a standard radioassay format using Protein A bound to Sepharose beads in 96-well membrane filtration plates to separate autoantibody-bound from free ³⁵S-labeled autoantigens.

Patient and control sera were tested for binding to [³⁵S]-labeled autoantigens in a radioimmunoassay format as follows. For each assay, an aliquot of in vitro translation reaction mixture (trichloroacetic acid (TCA)-precipitable material) was suspended in 50 μl of immunoprecipitation (IP) buffer containing:

For Actn2 assay: 20 mM Tris-HCl pH 7.4; 150 mM NaCl; 0.06 mM CaCl₂, 1 mM MgCl₂, 1.6 mM KCl, 1% (v/v) Triton X-100; 10 μg/ml aprotinin (Sigma, St Louis, USA).

For cardiac troponin I: 20 mM Tris-HCl pH 7.4, 150 mM NaCl, 1% (v/v)Triton X-100 ; 10 ug/ml aprotinin (Sigma, St Louis, USA).

For myosin and subfragments: 20 mM Tris-HCl pH 7.4, 150 mM NaCl, 1%

Nonidet P40 (Fluka); 10 ug/ml aprotinin (Sigma, St Louis, USA)

Approximately 50,000 counts per minute (cpm) of the in vitro translated protein were used in each RIA. Serum was then added to a final dilution of 1:25. The samples were incubated with shaking at room temperature for 1 hr prior to overnight incubation with shaking at 4° C.

After overnight incubation, 25 μl of protein A/G (50% A/8% G) Sepharose 4 Fast Flow beads (GE Healthcare, Piscataway, USA), prepared according to the manufacturer's protocol, were added to a 96 well plate (Unifilter, Whatman) and mixed with the reaction mixture. Incubation continued for 1 hour at 4° C. The protein A/G Sepharose-antibody complexes were then washed six times for 15 minutes in IP buffer at 4° C., after final wash beads were dry and resuspended in 100 ul scintillation fluid (Microscint 20, Perkim Elmer, USA) Immunoprecipitated radioactivity was evaluated in a LS 6500 multipurpose scintillation counter (Beckman Coulter, Fullerton, USA).

TABLE 2
Characteristics of Cardiac Autoantibody-Positive Patients

| | | | | Timing | | MYH6 | | | | | |
|---|---|---|---|---|---|---|---|---|---|---|---|
| Heart | Patient | Age | | from MI | | Fragments | | | | | |
| Disease | No | (y) | Gender | (y) | MYH6 | S1 | S2 | LMM | MYH7 | Actn2 | cTnI |
| T1D+ | 1 | 68 | F | u | − | − | − | − | − | + | − |
| MI+ | 2 | 56 | F | 7 | − | + | + | − | − | − | − |
| | 3 | 39 | F | 2 | − | − | − | − | − | + | − |
| | 4* | 59 | M | 4 | + | + | − | − | + | − | − |
| | 5 | 67 | F | 5 | − | + | − | − | − | − | − |
| | 6* | 56 | M | 7 | + | + | − | − | + | − | + |
| | 7 | 69 | M | 8 | − | + | − | − | − | − | − |
| | 8 | 57 | F | 5 | − | + | − | − | − | − | − |
| | 9 | 52 | M | u | − | + | − | − | − | − | − |
| | 10 | 19 | M | 5 | − | − | − | + | − | − | − |
| | 11 | 46 | F | 8 | − | + | − | − | − | − | − |
| | 12 | 66 | F | u | − | − | − | − | − | + | − |
| | 13* | 59 | M | 3 | + | + | − | − | + | − | − |
| | 14 | 55 | M | 3 | − | − | − | − | − | − | + |
| | 15 | 54 | F | 0.5 | − | + | − | − | − | − | + |
| T2D+ | 16 | 63 | M | 10 | − | − | − | + | − | − | − |
| MI+ | 17 | 57 | M | u | − | − | − | − | + | − | − |
| | 18 | 59 | F | 11 | − | + | − | − | − | − | + |
| Myo- | H10 | 36 | M | N/A | + | + | − | − | + | − | + |
| carditis, | H49 | 51 | F | | − | − | − | − | − | − | + |
| No | H99 | 50 | F | | − | + | + | − | − | − | − |
| diabetes | H102 | 21 | M | | − | − | − | + | − | − | − |
| | H104 | 33 | M | | + | − | + | + | + | nt | − |
| | H110 | 26 | M | | − | + | − | − | − | − | − |
| | M3 | 21 | M | | + | + | − | − | + | − | − |
| | M8 | 19 | M | | − | + | − | + | − | + | − |
| | P2 | 20 | F | | − | − | + | − | − | − | − |
| | P4 | 55 | M | | + | + | − | − | + | − | − |
| | P5 | 37 | F | | + | + | − | − | + | − | − |
| | P8 | 55 | M | | + | − | − | − | − | − | + |

Autoantibody reactivity was analyzed in a radioimmunoprecipitation assay format for full-length MYH6 (MyHC); S1, S2, and LMM fragments of MYH6; full-length MYH7; α-actinin-2 (Actn2), cardiac troponin I (cTnI).
−, negative for antibody reactivity;
+, positive for antibody reactivity;
u, timing of MI unknown;
N/A, not applicable;
nt, not tested;
*patient deceased.

These studies revealed that serum from 15/18 (83%) post-MT T1D patients tested positive to autoantibodies to 1 cardiac antigen, in contrast to 3/20 (15%) post-MI T2D patients and 4/78 (5.1%) healthy control subjects (T1D post-MI vs T2D post-MI, p=0.0002; T1D post-MI vs healthy controls, p<0.0001; T2D post-MI vs healthy controls, p=0.314) (Table 2 and FIG. 3).

The data also showed that post-MI T1D patients were more likely to have autoantibodies to troponin I and myosin heavy chain subfragment 1. In addition, data showed that the presence of autoantibodies to the S1 domain, but not to other domains, is not only a characteristic of T1D disease heart but also myocarditis, cardiomyopathy, and other forms of heart failure.

The data also demonstrate that testing for autoantibodies to multiple cardiac antigens (e.g., Actn2, cardiac myosin and TnI) has high positive predictive value for T1D ischemic heart disease (Table 2).

Finally, those patients who exhibited reactivity to multiple myosin isoforms (e.g., to MyH6 and MyH7) had an increased rate of mortality than did subjects who had reactivity to only one.

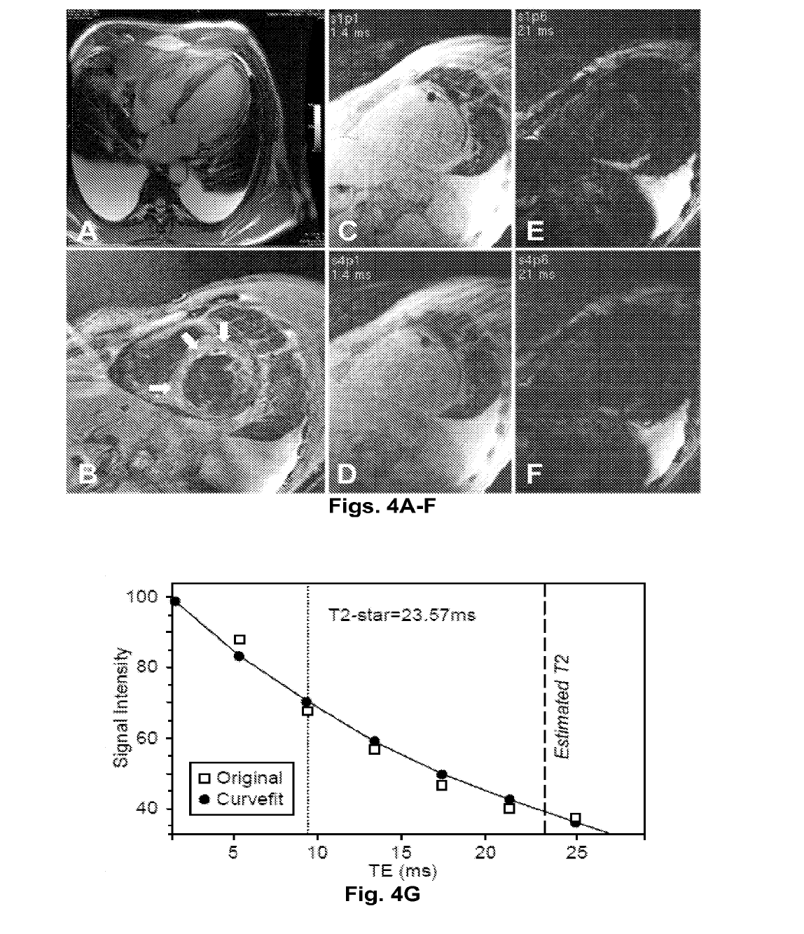

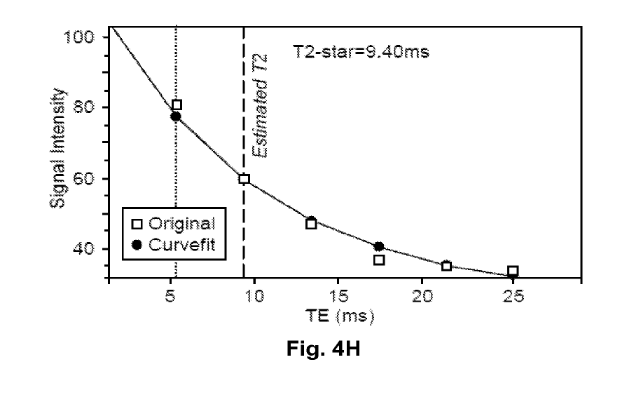

In addition, there is corroborating MRI evidence of inflammation in one of the T1D subjects who was cardiac autoantibody-positive (FIGS. 4A-H). Cine imaging (4A) showing dilated hypocontractile left ventricle with bilateral pleural effusion as a result of heart failure. There was evidence of chronic myocardial inflammation by T2-weighted fast spin-echo edema imaging (4B, arrows). In addition, myocardial inflammation was observed comparing T2* images before (4C and 4E, at echo times of 1.4 and 21 msec, respectively) and after (4D and 4F, at echo times of 1.4 and 21 msec, respectively) injection of the iron-oxide agent, Ferumoxytol. At 24 hr after injection, there was evidence of accumulation of iron-oxide in the myocardium, indicated by the decay constants of T2* (decay curves shown in 4G and 4H, 24 msec from before vs. 9.4msec after iron-oxide injection). This patient experienced sudden cardiac death less than 2 yr later.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for diagnosing the presence of myocardial autoimmunity in a subject, the method comprising:
providing a sample comprising serum of a subject; and
detecting the presence in the sample of one or both of:
autoantibodies that bind to alpha-actinin-2 (aActn2),
autoantibodies that bind to the S1 fragment of alpha-Myosin Heavy Chain (alpha-MHC-S1),
diagnosing the subject with myocardial autoimmunity based on the presence of autoantibodies to aActn2 and/or alpha-MHC-S1.

2. A method for selecting a treatment for a subject, the method comprising:
providing a sample comprising serum of a subject; and
detecting the presence or absence in the sample of one or both of:
autoantibodies that bind to alpha-Actinin-2 (aActn2),
autoantibodies that bind to the S1 fragment of alpha-Myosin Heavy Chain 6 (alpha-MHC6-S1),
wherein the presence of autoantibodies to aActn2 and/or alpha-MHC-S1 indicates that the subject has autoimmune myocarditis; and
selecting an immune-modulatory treatment for the subject who has autoimmune myocarditis.

3. A method for providing a prognosis or predicting risk of mortality in a subject who has ischemic heart disease the method comprising:
detecting the presence of autoantibodies that bind to alpha-Myosin Heavy Chain 6 (MyHC6); and
detecting the presence of autoantibodies that bind to alpha-Myosin Heavy Chain 7 (MyHC7);
wherein the presence of autoantibodies that bind to MyHC6 and autoantibodies that bind to MyHC7 indicates that the subject has a poorer prognosis or an increased risk of mortality as compared to a subject who does not have said autoantibodies.;

4. The method of any of claims 1 to 3, further comprising detecting the presence of autoantibodies to cardiac troponin I.

5. The method of claims 1 to 3, wherein the subject has an autoimmune disorder.

6. The method of claim 5, wherein the autoimmune disorder is type 1 diabetes or hypothyroidism.

7. The method of claims 1 to 3, wherein the subject has had ischemic heart disease.

8. The method of claim 7, wherein the ischemic heart disease is myocardial infarction (MI) or coronary artery disease (CAD).

9. The method of claim 8, wherein the subject experienced the ischemic heart disease at least one month prior to the provision of the sample.

10. The method of claim 8, wherein the subject experienced the ischemic heart disease at least one year prior to the provision of the sample.

11. The method of claim 2, wherein the treatment comprises administration of immunomodulatory therapies.

12. The method of claims 1 to 3, further comprising confirming the presence of myocarditis.

13. The method of claim 11, wherein the presence of myocarditis is confirmed by cardiac imaging.

14. The method of claims 1-13, further comprising administering an immunomodulatory therapy.

15. The method of claim 14, wherein the immunomodulatory therapy is administration of rituximab or anti-CD3 antibody.

16. A kit for use in the methods of claims 1-15, comprising one or more of the following panels of human autoantigens:
(i) Actn2 and TnI;
(ii) full-length MyHC6 and full-length MyHC7;
(iii) MyhC6-S1, MyhC6-S2, and MyhC6-LMM;
(iv) MyhC6-S1, MyhC6-S2, MyhC6-LMM full-length MyHC6, and full-length MyHC7; and/or
(v) Actn2, TnI, and one or more of MyhC6-S1, MyhC6-S2, MyhC6-LMM full-length MyHC6, and/or full-length MyHC7.

17. The kit of claim 16, wherein the autoantigens are labeled.

18. The kit of claim 17, wherein the autoantigens are radiolabeled.

19. The kit of claim 16, further comprising a reagent for detecting or purifying autoantigen/antibody complexes.

20. The kit of claim 19, wherein the reagent comprises one or both of protein A and protein G.

21. The kit of claim 19 or 20, wherein the reagent is bound to a bead.


SEQUENCE LISTING (6 sequences)

SEQ ID NO: 1 - DNA, 39 bp, Artificial Sequence
  synthetically generated primers
ttgcactcgagaattccgagatgaccgatgcccagatgg

SEQ ID NO: 2 - DNA, 39 bp, Artificial Sequence
  synthetically generated primers
taccacgcgtgcggccgctcacagcgtctctttgatgcg

SEQ ID NO: 3 - DNA, 36 bp, Artificial Sequence
  synthetically generated primers
ttgcacgtcgacaccatggccttcatgggggtcaag

SEQ ID NO: 4 - DNA, 39 bp, Artificial Sequence
  synthetically generated primers
taccactgcggccgctcacgccttgccctcctcctccag

SEQ ID NO: 5 - DNA, 36 bp, Artificial Sequence
  synthetically generated primers
ttgcacgtcgacaacatggagcagatcatcaaggcc

SEQ ID NO: 6 - DNA, 39 bp, Artificial Sequence
  synthetically generated primers
taccacgcgtgcggccgcaggttcccgaggcagtgtcac